(19) United States
(12) Patent Application Publication
(10) Pub. No.: US 2005/0153319 A1
(43) Pub. Date: Jul. 14, 2005

(54) Estrogen receptor gene variation and disease

(76) Inventors: David E. Housman, Newton, MA (US); Daniel Levy, Newton, MA (US); Michael E. Mendelsohn, Wellesley, MA (US); Amanda M. Shearman, Somerville, MA (US)

Correspondence Address: FISH & RICHARDSON PC, 225 FRANKLIN ST, BOSTON, MA, 02110, US

(21) Appl. No.: 10/982,726

(22) Filed: Nov. 4, 2004

Related U.S. Application Data
(60) Provisional application No. 60/517,425, filed on Nov. 4, 2003.

(51) Int. Cl. C12Q 1/68; C12P 19/34

(52) U.S. Cl. 435/6; 435/91.2

(57) ABSTRACT

Methods for determining the presence of polymorphisms in estrogen receptor genes and assessing an individual's risk for developing a condition are provided herein.

### CROSS-REFERENCE TO RELATED APPLICATIONS

This application claims the benefit of the filing date of U.S. Ser. No. 60/517,425, filed Nov. 4, 2003, the contents of which are hereby incorporated by reference in their entirety.

The work described herein was funded, in part, through grants from the National Institutes of Health (NIH), National Heart, Lung and Blood Institute (NHLBI) Specialized Center of Research in Ischemic Heart Disease (Grant Nos. P50 HL63494 and P50-HL63494; an NIH grant was also awared under grant number RO1-HL65230). The work is also supported by the NHLBI's Framingham Heart Study (NIH/NHLBI Contract N01-HC-38038 and NO1-HC-25195). The United States government may, therefore, have certain rights in the invention.

### TECHNICAL FIELD

This invention relates to human genetics, and more particularly to methods and compositions for detecting polymorphisms in an estrogen receptor gene in a subject and evaluating the risk for certain events, such as cardiovascular disease or other events associated with estrogen-based therapies.

### SUMMARY

The present invention features, inter alia, compositions and methods for assessing an individual's health and their risk for developing a condition (e.g., a disease (e.g., a cardiovascular disease (CVD)) or disorder) by determining the presence of a polymorphism in the individual's genome (e.g., in an intron, exon, or regulatory region (e.g., the promoter) of a gene encoding an estrogen receptor (e.g., an estrogen receptor alpha (ESR1) gene or an estrogen receptor beta (ESR2) gene)). The condition may be associated with exposure to an estrogen, whether endogenous or exogenously administered in the context of a study (e.g., a clinical trial or laboratory study), a therapeutic regime (e.g., a hormone-replacement therapy), or a contraceptive regime. Useful polymorphisms are not restricted to those that affect the encoded gene product in any particular way; the polymorphism can be any polymorphism that alters the expression level or function of the encoded protein (e.g., an encoded estrogen receptor). The polymorphism that is determined can also be one that is linked to a polymorphism that affects an encoded gene product (e.g., a polymorphism in linkage disequilibrium with a polymorphism that affects an encoded gene product).

More specifically, the invention features compositions and methods for determining the genotype of the c.454-397T>C polymorphism in the ESR1 gene in a sample obtained from an individual and thereby assessing the individual's risk for CVD. The presence of a polymorphism (e.g., a C allele in the c.454-397T>C genotype) indicates that the individual has an increased risk for CVD (e.g., myocardial infarction or atherothrombotic stroke), as does the presence of two C alleles. Alternatively, or in addition to, examining this polymorphism, the methods of the invention can be carried out by examining another polymorphism in ESR1 or a polymorphism in ESR2 (e.g., a polymorphism in linkage disequilibrium with the c.454-397T>C polymorphism). ESR1 polymorphisms that can be examined include: c.454-351A>G; c30T>C; c.30T>C, c.975c>G. Polymorphisms in the ESR1 and ESR2 genes can be found, e.g., in the SNP database of the National Center for Biotechnology Information on the worldwide web at ncbi.nlm.nih.gov/SNP. Polymorphisms of ESR2 that can be examined include those listed in the SNP database under identification number rs1256031, rs1256034, rs1256059.

The sample can be a sample of nucleic acids (i.e., pure or substantially pure nucleic acids (e.g., genomic DNA or mRNA)) or a sample containing nucleic acids and other materials (e.g., other biological materials or reagents). Nucleic acids obtained from the individual may be further manipulated before the evaluation begins. For example, the evaluation of genotype can be carried out using cDNA made from nucleic acids obtained from the individual or using a PCR product amplified from a nucleic acid of the individual.

We may use the term “individual” or “patient” below, as the methods of the invention can be carried out on individuals that are healthy, or apparently healthy, or individuals who have CVD or some other ailment. Where an individual is healthy, the compositions and methods of the invention can be used to assess the risk of developing CVD; where an individual has already developed CVD (e.g., the individual has had an MI or stroke), the compositions and methods of the invention can be used to assess the risk of recurrence. A patient may have been examined and diagnosed as having such a condition or may simply be a person experiencing a sign or symptom of such a condition (i.e., the individual need not be definitively diagnosed to be a candidate for the evaluative methods of the present invention). More specifically, an individual or patient subjected to a method of the invention can be a person or laboratory animal.

The CVD may be any one or more of a variety of conditions. For example, the CVD can be atherosclerosis, acute myocardial infarction, angina pectoris, venous thrombosis, coronary insufficiency, coronary heart disease death, hypertension, hypercholesterolemia, intermittent claudication, or stroke (also known as a cerebrovascular accident; e.g., atherothrombotic stroke).

The methods described herein (e.g., wherein an individual's ESR1 genotype is determined) can also be used to assess an individual's risk for developing metabolic syndrome, which is characterized by central obesity, atherogenic dsylipidemia, raised blood pressure, insulin resistance or glucose intolerance, prothrombic state, and proinflammatory state (e.g., elevated C-reactive protein in the blood). The presence of a C allele at the c454-397T>C polymorphism can indicate increased risk for metabolic disorder.

The methods that can be used to determine the genotype (i.e., methods of “genotyping”) are described further below. We note here that determining an individual's genotype can include providing nucleic acid obtained from the individual and exposing the nucleic acid (e.g., DNA (e.g., genomic DNA)), to a restriction endonuclease that recognizes a polymorphism in the nucleic acid (e.g., Pvu II, which recognizes a polymorphism in the ESR1 gene) under conditions and for a time sufficient to allow the endonuclease to cleave the nucleic acid. Optionally, the nucleic acid obtained from a patient can be further manipulated (e.g., amplified by PCR). Alternatively, the genotyping can be carried out by a hybridization method or by sequencing the nucleic acid. “Genotyping” refers to any method of evaluating genetic material (e.g., the sequence of an intron, exon, or regulatory region (e.g., a promoter) in an estrogen receptor gene). In the context of the present invention, it refers to any method of determining the type and number of alleles and/or polymorphisms present in an estrogen receptor-encoding gene such as ESR1.

The nucleic acid assessed can be obtained from any of the individual's tissues or a sample of bodily fluids. Conveniently, the nucleic acid may be obtained from a blood sample. We expect that the methods of genotyping will typically be carried out directly on genomic DNA, but other methods, which require other types of nucleic acids (e.g., cDNA or mRNA) or that require proteins are also within the scope of the present invention. The nucleic acids, proteins, or other materials used in the assays need only be purified to an extent that allows the assays to be successfully carried out.

Genotyping, by any method, can be carried out in concert with an assessment of other factors. For example, estrogen receptor genotyping can be carried out in concert with assessment of other genomic factors (e.g., assessment of other polymorphisms, serological markers, immunological markers, or gene or protein expression profiles, which may or may not indicate a predisposition for CVD) or factors related to the patient's lifestyle or health. For example, the invention features methods of evaluating a patient's risk for cardiovascular disease (e.g., myocardial infarction or atherothrombotic stroke) by determining the presence of a polymorphism in an estrogen receptor gene (e.g., the ESR1 gene). The methods can include determining the genotype of the c.454-397T>C polymorphism in the ESR1 gene and/or another polymorphism in the ESR1 gene and/or a polymorphism in ESR2 in a nucleic acid sample obtained from an individual and determining whether the patient: (a) regularly smokes cigarettes or uses another tobacco product; (b) is sedentary (or exercises less than a recommended amount); (c) has high blood pressure; (d) has elevated blood cholesterol levels; (e) has genetic relatives who have cardiovascular disease; and/or (f) is experiencing a sign or symptom of a cardiovascular disease. The more of these factors the patient experiences, the greater their risk for cardiovascular disease or stroke. The patient's weight or body mass index (typically calculated as weight in kilograms divided by the square of height in meters) can also be considered. Similarly, the genotyping methods of the invention, as well as methods in which genotype and other factors are considered together, can include an analysis of the patient's genetic relatives. For example, one can determine the genotype of the allele in a nucleic acid sample obtained from a genetic relative of the patient. While it is useful to assess risk per se, the methods of the invention can also be carried out in order to predict how a given patient is likely to respond to a therapeutic regime and/or to determine whether that patient is a good candidate for a therapeutic regime. For example, the invention features a method of predicting how a patient will respond to an anti-atherosclerotic therapy by determining the genotype of a polymorphism described herein (e.g., the c.454-397T>C polymorphism of ESR1) in a sample obtained, directly or indirectly, from the patient. Here too, the presence of the polymorphism (e.g., the C allele of the c.454-397T>C polymorphism) indicates that the patient is likely to benefit from therapy with an anti-atherosclerotic agent, as does the presence of two such alleles. Here too, the genotyping can be combined with an assessment of any of the other factors listed above (e.g., smoking or another factors). The precise therapy can be any of those available. For example, the anti-atherosclerotic therapy can be a lipid-lowering therapy (e.g., statin therapy), an anti-platelet therapy (e.g., aspirin, clopidogrel, or ticlopidine therapy), an anti-coagulant therapy (e.g., heparin therapy) or any other type of therapy indicated for treatment or prevention of a sign or symptom of a cardiovascular condition.

Another application of the present methods is in the context of hormone or hormone-based therapeutics. Women who take estrogen-containing medications (as, for example, contraceptives) may increase their risk of cardiovascular disease or stroke (as well as experience other adverse side effects). The methods of the present invention can be carried out to determine whether a given individual may be more prone to experiencing these adverse effects (and may, therefore, be inclined to choose an alternative therapeutic or form of birth control). As described above, these methods include genotyping a polymorphism of the ESR1 and/or ESR2 gene (e.g., the c.454-397T>C polymorphism of ESR1) in a sample obtained from the individual.

Similarly, the methods of the invention can be carried out in the context of hormone replacement therapy. By determining an individual's genotype with respect to the ESR1 or ESR2 gene, a patient and her physician are better able to weigh the risks and benefits of commencing such therapy.

The methods of the invention also encompass methods for determining whether a patient is likely to respond positively to a therapeutic agent. The methods can be carried out by administering the agent to a group of patients and determining which of the patients responds positively. Either before, during, or after the time the patients' response is evaluated, polymorphisms in an estrogen receptor gene (e.g., ESR1 or ESR2), including one or more of the polymorphisms described herein, are examined. The presence or absence of the polymorphism is then correlated with clinical outcome. Once a correlation is established, one can determine whether any given patient has the polymorphism and, thereby, determine whether that patient is likely to respond positively to the therapeutic agent. Where the presence of the polymorphism correlates with poor clinical outcome, one can conclude that a patient having that polymorphism is unlikely to respond positively to the therapeutic agent; where the presence of the polymorphism correlates with good clinical outcome, one can conclude that a patient having that polymorphism is likely to respond positively to the therapeutic agent. The agent can be any type of therapeutic agent (e.g., a nucleic acid (whether all or part of a coding or antisense sequence), peptide or protein (including an antibody or a fragment thereof (e.g., a single-chain antibody), or a small molecule. The agent can be considered a pharmaceutical agent or a nutraceutical (e.g., a dietary supplement such as a vitamin, mineral, or other substance (e.g., an oil)). The agent can be administered according to established protocols for the agent or in the manner prescribed during a clinical trial.

The compositions of the invention include probes and primers suitable for determining the genotype of polymorphisms within an estrogen receptor gene (e.g., the c.454-397T>C polymorphism of ESR1, or another polymorphism of ESR1 or of ESR2. The primers can be, for example, about 10 to 2000 nucleotides long (e.g., about 10 to 500, 10 to 100, 10 to 50, or 14 to about 30 nucleotides long) and will have, or will include, a sequence that is sufficiently complementary to the ESR1 or ESR2 sequence to hybridize with the complimentary sequence under stringent or very stringent conditions. The terms “stringent” or “very stringent” describe conditions for hybridization and washing. Guidance for performing hybridization reactions can be found in Current Protocols in Molecular Biology, John Wiley & Sons, N.Y. (1989) 6.3.1.-6.3.6. Aqueous and nonaqueous methods are described in that reference and either can be used.

The invention also includes kits that include reagents to facilitate the methods described herein. For example, the invention features a kit that includes one or more ESR1- or ESR2-specific primers (i.e., oligonucleotides having a sequence that is sufficiently complementary to the ESR1 or ESR2 gene sequence that they can be used, alone or in combination (as in PCR) to identify the genotype of an allele of a polymorphism (e.g., a c.454-397T>C polymorphism). The primers can be packaged with other useful reagents, such as buffers, enzymes, and nucleotides, which can be used to amplify or sequence an ESR1- or ESR2-containing nucleic acid sample obtained from an individual. The kit can include instructions for genotyping (e.g., written instructions and/or audio and/or videotaped instructions).

In another embodiment, the invention features computer-readable databases that include a plurality of records. Each record includes (a) a first field that includes information reflecting the genotype of one or both alleles of a c.454-397T>C polymorphism of the ESR1 gene of a human subject, and (b) a second field that includes information concerning a cardiovascular parameter of the subject. As in the methods of the invention, the subject can be of any age, and the database can include records of patients from different age groups (e.g., records of patients under 20 years old; under 30; under 40; and so forth). The information contained within the fields can be obtained in any order (e.g., the information reflecting the genotype can be obtained first). However, to help ensure the integrity of the database, the information should be obtained independently (or “blindly”). The database can also include a field comparing the cardiovascular parameter to a clinical outcome associated with the parameter (e.g., an MI or stroke). The cardiovascular parameter can include any of the parameters referenced above, including high blood pressure, a high blood cholesterol level (or other measurements of lipoproteins (e.g., HDL and/or LDL levels or ratios), an abnormal electrographic profile, or angina. Of course, information concerning multiple parameters can be included. The number of records can be, but is not necessarily, great. For example, a useful database can include at least 50, 100, 250, 500, 1000, 1500, 1800, 2000, 2500, 5000, 6000, or 7500 records.

As the methods of the invention can be carried out on an individual of any age, they can be carried out on young people, who may benefit greatly from taking preventative measures early (whether those measures include a change in lifestyle or resort to medication). For example, the method can be carried out on an individual that is less than about 20 years old; less than about 30 years old; less than about 40 years old; less than about 50 years old; or less than about 60 years old.

The details of one or more embodiments of the invention are set forth in the accompanying drawings and the description below. Other features, objects, and advantages of the invention will be apparent from the description and drawings, and from the claims. All patents, patent applications, and publications referenced herein are incorporated by reference in their entirety.

### BRIEF DESCRIPTION OF THE DRAWINGS

FIG. 1 is a table listing the characteristics of Framingham Offspring Cohort participants included in and excluded from analysis in the study described herein.

FIG. 2 is a table listing the characteristics of the study participants by ESR1 c.454-397T>C genotype.

FIG. 3 is a table listing the genotype and allele frequencies of ESR1 polymorphisms in the study participants.

FIG. 4 is a table listing the results of the association test for ESR1 c.454-397T>C genotype and cardiovascular events in 1739 unrelated men and women from the Framingham Offspring Cohort.

FIG. 5 is a graph depicting the association of ESR1 c.454-397T>C polymorphism with risk of myocardial infarction in 1739 unrelated men and women from the Framingham Heart Study. P values, odds ratios and 95% confidence intervals (error bars) are shown for a genotype model, and a recessive “C allele” model, after adjustment for age, sex, body mass index, hypertension, diabetes mellitus, total cholesterol, HDL-cholesterol, and smoking. In the genotype model P=0.005 for T7 vs. TC; P<0.001 for TC vs CC; and P=0.005 for TT vs CC.

FIG. 6 is a table listing the results of the association test for ESR1 c.454-397T>C genotype and cardiovascular events in 875 unrelated men from the Framingham Offspring Cohort.

FIG. 7 is a table listing the results of the association test for ESR1 c.301T>C, ESR1 c.454-351A>G, and ESR1 c.975C>G genotypes and cardiovascular events in 1739 unrelated Framingham Offspring Cohort participants.

FIG. 8 is a graph depicting the association of ESR1 c.454-397T>C polymorphism with MI in men from five studies. Combined results from an adjusted fixed effects model are depicted. OR=odds ratio.

### DETAILED DESCRIPTION

Atherothrombotic cardiovascular diseases (CVD) such as myocardial infarction and stroke are multifactorial disorders with substantial heritable components. Genetic constitution may contribute to conditions such as hypertension, diabetes mellitus, and hypercholesterolemia and may act through other indirect routes to alter susceptibility to CVD events (Nora et al. Circulation 61:503-508, 1980; Marenberg et al. N. Engl. J. Med. 330:1041-1046, 1994). A body of epidemiological evidence suggests that endogenous estrogen has a protective role in the development of cardiovascular diseases. However, in several clinical trials, combination hormone replacement therapy was associated with increased incidence of myocardial infarction and stroke (Rossouw et al. JAMA. 288:321-333, 2002; Waters et al. JAMA. 288:2432-2440, 2002; and Ferrara et al. Circulation 107:43-48, 2003). The methods described herein can be used, alone or in combination with other methods and information, to evaluate an individual patient's risk of cardiovascular disease (or any other disease or condition that may follow as an undesirable result) should the patient begin a therapeutic regime in which estrogen (or any drug substance that stimulates estrogen receptor activity) is administered.

Estrogens and estrogen receptors have important physiological roles in men as well as in women. Accordingly, the methods described herein can be applied to evaluate risk in either sex (similarly, the databases described can be generated from data collected from both men and women or either men or women; the diagnostic reagents can be used with tissue samples collected from both men and women; and so forth). There are two known estrogen receptors: estrogen receptor α (ESR1) and estrogen receptor β (ESR2). Both receptors are expressed in a wide range of tissues including macrophages, vascular smooth muscle and vascular endothelial cells (Mendelsohn and Karas. N. Engl. J. Med. 340:1801-1811, 1999). While the compositions and methods described herein are focused on ESR1, polymorphisms in other genes, including ESR2, or polymorphisms in linkage disequilibrium with an ESR1 polymorphism, can be assessed as well.

Estrogen receptors are believed to regulate gene expression by both estrogen-dependent and estrogen-independent mechanisms that result in activation of transcription. There have been several genetic association studies, each limited to a few hundred individuals, of ESR1 variants in relation to coronary artery disease (Matsubara et al., Arterioscler. Thromb. Vasc. Biol. 17:3006-3012, 1997; Kunnas et al., BMJ. 321:273-274, 2000; Lu et al., Arterioscler. Thromb. Vasc. Biol. 22:817-823, 2002; Evangelopoulos et al., Clin. Chim. Acta. 331:37-44, 2003; Petrovic and Peterlin, Cardiology 99:163-165, 2003), coronary artery wall atherosclerosis (Lehtimaki et al., J. Mol. Med. 80:176-180, 2002), and variation in high density lipoprotein-cholesterol or E-Selectin level in response to estrogen replacement therapy (Herrington et al., N. Engl. J. Med. 346:967-974, 2002; Herrington et al., Circulation.105:1879-1882, 2002). Three of the four positive association findings examined the IVS1-401 T/C variant (also known as c.454-397T>C, and the PvuII restriction site) in intron 1 of ESR1 (Lehtimaki et al., J. Mol. Med. 80:176-180, 2002; Herrington et al., N. Engl. J. Med. 346:967-974, 2002; Herrington et al., Circulation 105:1879-1882, 2002). The C allele but not the T allele forms part of a potential binding site for the myb family of transcription factors (Herrington et al., Circulation.105:1879-1882, 2002) which are among the many genes whose transcription is activated by estrogen (Jeng et al., Endocrinology 139:4164-4174, 1998). Thus, transcriptional regulation of a gene containing the C allele may be different than other alleles (e.g., transcription of C allele-containing genes may be increased).

The present invention is based, in part, on our discovery of the impact of genetic variation in estrogen receptor alpha (ESR1) on risk for disease, including cardiovascular disease and stroke. Accordingly, the invention features methods and compositions for determining the risk of cardiovascular disorders and stroke in a subject (e.g., a human patient) by determining the genotype of a c.454-397T>C polymorphism of an estrogen receptor alpha (ERS1) gene in a tissue sample obtained from that subject. The rare form of this polymorphism is associated with increased risk for cardiovascular disorders, including myocardial infarction (MI) in human subjects. Moreover, analysis of this polymorphism can be used in evaluating risk for certain cardiovascular disorders and stroke and for determining appropriate therapeutic regimens (e.g., those most likely to succeed, or the most likely to succeed with minimal side effects) for subjects based on their c.454-397T>C polymorphism genotype.

The Estrogen Receptor Alpha Gene and the c.454-397T>C Polymorphism

The c.454-397T>C allele, which we refer to herein as the “C allele” (also known as the IVS1-401 T/C variant, the PvuII restriction site, and the PvuII polymorphism), is to a single nucleotide polymorphism in an intron of the ESR1 gene. This polymorphism is 397 nucleotides upstream of the exon beginning with nucleotide 454 of the coding sequence according to version 12 of the chromosome reference sequence in GenBank® under GenBank® Accession No. NT_{—}023451. The T allele of ESR1 c.454-397T>C is susceptible to cleavage with the restriction endonuclease, PvuII, and has been termed the p allele in some previous reports; likewise the C allele, in which the PvuII site is absent, has been termed the P allele. This single nucleotide polymorphism (SNP) is also listed the SNP database under identification number rs2234693 (on the worldwide web at ncbi.nlm.nih.gov/SNP). The SNP and genomic sequences surrounding the SNP are shown in Table 1. As described herein, a sequence including the ESR1 gene SNP can be detected in any of the diagnostic or predictive methods described herein, and the sequence can be detected by the reagents within the kits assembled for that purpose.

Genetic Information

ESR1 locus genetic information can be obtained by, for example, evaluating genetic material (e.g., genomic DNA or nucleic acids amplified from the genomic DNA) from a subject. Genetic information refers to any information about nucleic acid sequence content at one or more nucleotide positions. Such information can include, for example, an indication about the presence or absence of a particular polymorphism (e.g., one or more nucleotide variations). Exemplary polymorphisms include a single nucleotide polymorphism (SNP), a restriction site or restriction fragment length polymorphism, an insertion, an inversion, a deletion, a repeat (e.g., a trinucleotide repeat, a retroviral repeat), and so forth. Thus, the term “polymorphism” generally refers to any variation in sequence at a given position or region of nucleic acid sequence between individuals in a population (e.g., humans). As noted, variations include nucleotide substitutions (e.g., transitions and transversions), insertions, deletions, inversions, and other rearrangements. A variation can encompass one or more nucleotide positions in a reference sequence (e.g., a “first” sequence) that are absent, altered, inverted, or otherwise rearranged in another sequence (e.g., a “second” sequence). Some exemplary polymorphisms cause one or more changes in the amino acid sequence of an encoded protein and can affect cellular events such as transcription, translation, splicing, mRNA or protein stability, mRNA or protein localization, chromatin organization, and so forth. Still other exemplary polymorphisms are silent or are only manifest under particular circumstances. Even completely silent markers are useful. For example, they may be tightly linked to a marker that is causative of a particular property. Typically, a polymorphic marker described herein is an inherited variant, but may also arise through a spontaneous recombination event or by artificial means (e.g., by a targeted genetic manipulation). A “functional polymorphism” is a polymorphism which itself conditions a phenotype, as opposed to a polymorphism which flanks or is linked to a region.

Methods of Evaluating Genetic Material

There are numerous methods for evaluating genetic material to provide genetic information. These methods can be used to evaluate a polymorphism in the ESR1 gene (e.g., a c.454-397T>C polymorphism). For example, the genotype of the c.454-397T>C polymorphism can be determined by digesting genomic DNA obtained from a subject with the restriction endonuclease Pvu II. Optionally, the DNA can be amplified by PCR as described in Yaich et al. (Cancer Res. 52(1):77-83, 1992) before it is digested or otherwise analyzed (e.g., sequenced). Alternatively, or in addition, nucleic acid samples can be analyzed using biophysical techniques, such as hybridization and electrophoresis, sequencing (sequencing using polymerases, e.g., DNA polymerases and variations thereof, such as single base extension technology, described in, for example, U.S. Pat. Nos. 6,294,336; 6,013,431; and 5,952,174), and combinations-thereof. For example, hybridization of sample nucleic acids to nucleic acid microarrays can be used to evaluate sequences in a nucleic acid population and to evaluate genetic polymorphisms (accordingly, as noted above, the invention features a nucleic acid microarray comprising a sequence to which an ESR1 c.454-397T>C polymorphism would bind). Other hybridization based techniques include sequence specific primer binding (e.g., PCR or ligase chain reaction (LCR)); Southern analysis of DNA; Northern analysis of RNA (e.g., mRNA); fluorescent probe based techniques (see, e.g., Beaudet et al., Genome Res. 11:600-608, 2001); and allele specific amplification. Electrophoretic techniques include capillary electrophoresis and Single-Strand Conformation Polymorphism (SSCP) detection (see, e.g., Myers et al. Nature 313:495-8, 1985 and Ganguly, Hum Mutat. 19:334-342, 2002). Other biophysical methods include denaturing high pressure liquid chromatography (DHPLC). These and other techniques are well known to one of ordinary skill in the art.

Allele specific amplification technology that depends on selective PCR amplification can be used to obtain genetic information. Oligonucleotides used as primers for specific amplification may carry the mutation of interest in the center of the molecule (so that amplification depends on differential hybridization; Gibbs et al., Nucleic Acids Res. 17:2437-2448, 1989) or at the extreme 3′ end of one primer where, under appropriate conditions, mismatch can prevent, or reduce polymerase extension (Prossner, Tibtech 11:238, 1993). In addition, it is possible to introduce a restriction site in the region of the mutation to create cleavage-based detection (Gasparini et al., Mol. Cell Probes 6:1,1992). In amplification techniques, one can use Taq ligase in the amplification process (Barany, Proc. Natl. Acad. Sci USA 88:189, 1991). In such cases, ligation will occur only if there is a perfect match at the 3′ end of the 5′ sequence making it possible to detect the presence of a known mutation at a specific site by looking for the presence or absence of amplification. As indicated above, the invention features compositions (e.g., ESR1-specific primers) useful in identifying a c.454-397T>C polymorphism (whether by acting as probes to detect the polymorphism or as primers to amplify the relevant sequence) and kits containing them.

The amplification-based methods, such as those that include PCR can be carried out using guidance from laboratory manuals, if necessary, or publications (see, e.g., Saiki et al., Science 230:1350-1354, 1985; for methods that include LCR, one could consult Wu et al., Genomics 4:560-569, 1989 and Barringer et al., Gene 89:117-122, 1990). For guidance on transcription-based methods utilizing RNA synthesis by RNA polymerases (to amplify nucleic acid) see, e.g., U.S. Pat. No. 6,066,457; U.S. Pat. No. 6,132,997; and U.S. Pat. No. 5,716,785; Sarkar et al. (Science 244:331-334,1989); and Stofler et al. (Science 239:491,1988); for nucleic acid sequence-based amplification, see U.S. Pat. Nos. 5,130,238; 5,409,818; and 5,554,517; for rolling circle amplification (RCA) see U.S. Pat. Nos. 5,854,033 and 6,143,495; and for strand displacement amplification (SDA) see U.S. Pat. Nos. 5,455,166 and 5,624,825. Amplification methods can be used in combination with other techniques (e.g., hybridization-based and/or sequence-based techniques).

Mass spectroscopy (e.g., MALDI-TOF mass spectroscopy) can also be used to detect nucleic acid polymorphisms. For example, with the MassEXTEND™ assay (SEQUENOM, Inc.), selected nucleotide mixtures, missing at least one dNTP and including a single ddNTP one can extend a primer that hybridizes near a polymorphism. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides can be positioned on a nucleic acid array. Each oligonucleotide can contain the polymorphic nucleotide, wherein the position of the polymorphic nucleotide varies among members of the set. The extent of hybridization as a function of position and hybridization to oligonucleotides specific for the other allele can be used to determine whether a particular polymorphism is present. See, e.g., U.S. Pat. No. 6,066,454.

If desired, hybridization probes can include one or more additional mismatches to destabilize duplex formation and sensitize the assay (such mismatched probes that specifically bind ESR1 are within the scope of the invention). The mismatch may be directly adjacent to the query position, or within about 10 (e.g., 2, 3, 4, 5, or 7) nucleotides of the query position. Hybridization probes can also be selected to have a particular Tₘ (e.g., between 45-60° C., 55-65° C., or 60-75° C.). In a multiplex assay, Tₘ's can be selected to be within several degrees of one another (e.g., 5, 3, or 2° C. of each other).

It is also possible to directly sequence the nucleic acid for a particular genetic locus by, for example, amplification and sequencing, or amplification, cloning and sequencing. High throughput automated (e.g., capillary or microchip based) sequencing apparati can be used. In still other embodiments, the sequence of an ESR1 protein is analyzed to infer its genetic sequence (e.g., to determine the presence of a polymorphism that causes an amino acid change in the polypeptide). Methods of analyzing a protein sequence include protein sequencing, mass spectroscopy, sequence/epitope specific immunoglobulins, and protease digestion. Any combination of the above methods can also be used.

Cardiovascular Parameters

A variety of criteria, including genetic, biochemical, and physiological criteria, can be used to evaluate cardiovascular parameters in a subject. For example, symptoms of cardiovascular diseases (e.g., chest pain associated with myocardial infarction or angina and the speech and motor impairments associated with stroke) are known to one of ordinary skill in the art and can be assessed and correlated with a given polymorphism. See, e.g., Section 16 of The Merck Manual (Beers and Berkow, eds. Merck & Co., 17^{th} Ed., 1999) for such symptoms. Symptoms of cardiovascular diseases include chest pain (or pain referred to the back or arm), weakness, palpitations, nausea and light-headedness. Risk factors for cardiovascular disease can also be considered cardiovascular parameters and used in the methods of analysis described herein. Risk factors for cardiovascular disease include smoking, high cholesterol, diabetes mellitus, excessive alcohol consumption, advanced age, and obesity. A cardiovascular parameter, whether a symptom, sign, risk factor, or other factor associated with cardiovascular disease, can include qualitative or quantitative information and may be objective or subjective. An example of quantitative information is a numerical value of one or more dimensions, e.g., a concentration of cholesterol in the blood. Qualitative information can include a personal assessment or observation, e.g., a physician's comments or a binary response (“yes”/“no”) from the patient or another source and so forth. A cardiovascular parameter includes information indicating whether or not a patient has been diagnosed with a cardiovascular disease or a symptom of a cardiovascular disease, including results of diagnostic tests, e.g., results of an electrocardiographic test indicating aberrations characteristic of myocardial infarction.

Techniques to detect biochemical abnormalities in a sample from a subject, including abnormalities that are associated with a cardiovascular parameter, include cellular, immunological, and other biological methods known in the art. For general guidance, see, e.g., techniques described in Sambrook & Russell, Molecular Cloning: A Laboratory Manual, 3^{rd} Edition, Cold Spring Harbor Laboratory, N.Y., 2001; Ausubel et al., Current Protocols in Molecular Biology (Greene Publishing Associates and Wiley Interscience, N.Y., 1989); Harlow, E. and Lane, D. Antibodies: A Laboratory Manual, Cold Spring Harbor Laboratory Press, Cold Spring Harbor, N.Y., 1988), and updated editions thereof. More specifically, agents (e.g., metabolites) that are associated with cardiovascular disease can be detected by a variety of techniques, including enzyme-coupled assays and nuclear magnetic resonance (NMR). Where the agent affects a precursor, the precursor can be labeled and the level quantitated or otherwise assessed. For example, antibodies, other immunoglobulins, and other specific binding ligands can be used to detect a protein or biological agent (e.g., a lipid) associated with cardiovascular disease. For example, one or more specific antibodies can be used to detect a protein associated with cardiovascular disease in a sample. Various formats are possible. For example, one can detect the protein using ELISAs, fluorescence-based assays, Western blots, or protein arrays. Methods of producing polypeptide arrays are known in the art (see, e.g., De Wildt, et al., Nature Biotech. 18:989-994, 2000; Lueking, et al., Anal. Biochem. 270:103-111, 1999; Ge, H. Nucleic Acids Res. 28:e3, I-VII, 2000; MacBeath and Schreiber, Science 289:1760-1763, 2000; and WO 99/51773A1). Proteins can also be analyzed using mass spectroscopy, chromatography, electrophoresis, enzyme interaction or by using probes that detect post-translational modification.

Polymorphisms can be detected in cells that have been removed from a patient (e.g., removed by surgery or otherwise extracted), or that are in a fluid sample (e.g., a sample of blood, urine, or saliva). Where polymorphisms are assessed in order to determine a patient's risk of cardiovascular disease or the risk they would encounter when exposed to estrogens, the patient will obviously be living. However, where data is being gathered for storage in a database, the patient can be living or deceased. Polymorphisms within one or more genes can then be evaluated, by, for example, the techniques described above.

Nucleic acid arrays are useful for profiling multiple nucleic acid species in a sample. A nucleic acid array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In one embodiment, a patient's c.454-397T>C genotype is determined, and the genotype of a polymorphism of a second gene associated with cardiovascular disease is determined. For example, the genotype of connexin 37, plasminogen-activator inhibitor type 1, and/or stromelysin-1 can be determined. These genes have polymorphisms associated with cardiovascular disease as described in Yamada et al. (New Engl. J. Med. 347(24): 1916-1923, 2002). Other gene polymorphisms which can be examined are shown in Table 1 of Yamada et al. New Engl. J. Med. 347(24):1916-1923, 2002.

As noted above, information about a cardiovascular parameter can be recorded and/or stored in a computer-readable format. Typically, the information is linked to information about the subject and may also be associated (directly or indirectly) with information about the identity of one or more nucleotides in the subject's genes (e.g., nucleotides in the ESR1 gene).

Therapeutic Approaches

Both prophylactic and therapeutic methods of treatment can be specifically tailored or modified, based on knowledge obtained from analysis of a c.454-397T>C polymorphism genotype. This can allow a physician or other healthcare professional to prescribe prophylactic or therapeutic treatments more effectively. For example, the methods of the invention will support aggressive prophylactic treatment for patients at higher risk for cardiovascular disease and allow patients at lower risk to avoid treatment or select other treatment regimes (e.g., non-estrogen-based regimes). In particular, a drug that affects a cardiovascular parameter (e.g., cholesterol levels or blood pressure) can be prescribed as a function of the subject's c.454-397T>C genotype. A subject having a CC genotype of the c.454-397T>C polymorphism may be prescribed more aggressive therapy to treat and/or prevent the development of cardiovascular disease. Alternatively, patients at high risk for cardiovascular disease can be placed in a monitoring program to monitor for progression of cardiovascular disease (in which event the patient may be prescribed a medication).

### EXAMPLES

### Example 1

### Association of the ESR1 c.454-397T>C Polymorphism with Myocardial Infarction

The following study was undertaken to investigate whether the ESR1 c.454-397T>C polymorphism is associated with risk for CVD. This is a prospective study of 1739 unrelated men and women, from the population based offspring cohort of the Framingham Heart Study, who were followed from 1971 to 1998. The main outcome measures we investigated were: total atherosclerotic CVD (defined as recognized or unrecognized myocardial infarction, angina pectoris, coronary insufficiency, or intermittent claudication) (n=178); major atherosclerotic CVD (defined as recognized acute myocardial infarction, coronary insufficiency, coronary heart disease death, or atherothrombotic stroke) (n=83); and recognized acute myocardial infarction (n=59).

We found that twenty percent of subjects (n=352) were homozygous for the ESR1c.454-397C allele. After adjustment for covariates (age, sex, body mass index, hypertension, diabetes mellitus, total cholesterol, HDL-cholesterol, and cigarette smoking) the c.454-397CC genotype was significantly associated with major CVD, with an odds ratio of 2.0 (95% confidence interval [CI], 1.3-3.2; p=0.004) compared to the TC or TT genotypes. Subjects with the CC genotype had 3.0 fold greater odds of myocardial infarction (95% CI, 1.7-5.2; p=0.0001) compared to those with the TC or TT genotype. The results remained significant when analyses were restricted to men.

The conclusion of this study is that individuals with the common ESR1 c.454-397CC genotype have a substantial increase in risk of myocardial infarction. These findings support the importance of estrogen receptors in CVD susceptibility, especially in men. Estrogen receptor variation may explain recent conflicting data regarding the effects of hormone replacement therapy (HRT) on CVD susceptibility in women.

Study Sample

The Framingham Heart Study began in 1948 with enrollment of 5209 men and women aged 28 to 62 years from Framingham, Mass. The participants were examined every two years. Details of study design and selection criteria are described elsewhere (Dawber et al. Am. J. Public Health. 41:279-286, 1951; Dawber et al., Annals of the New York Academy of Sciences. 107:539-556, 1963). In 1971, 5124 offspring of original cohort members and spouses of offspring were recruited. After their initial evaluation, these individuals underwent repeat examinations approximately every four years thereafter. Details of the Framingham offspring cohort selection criteria have been described (Kannel et al., Am. J. Epidemiol. 110:281-290, 1979).

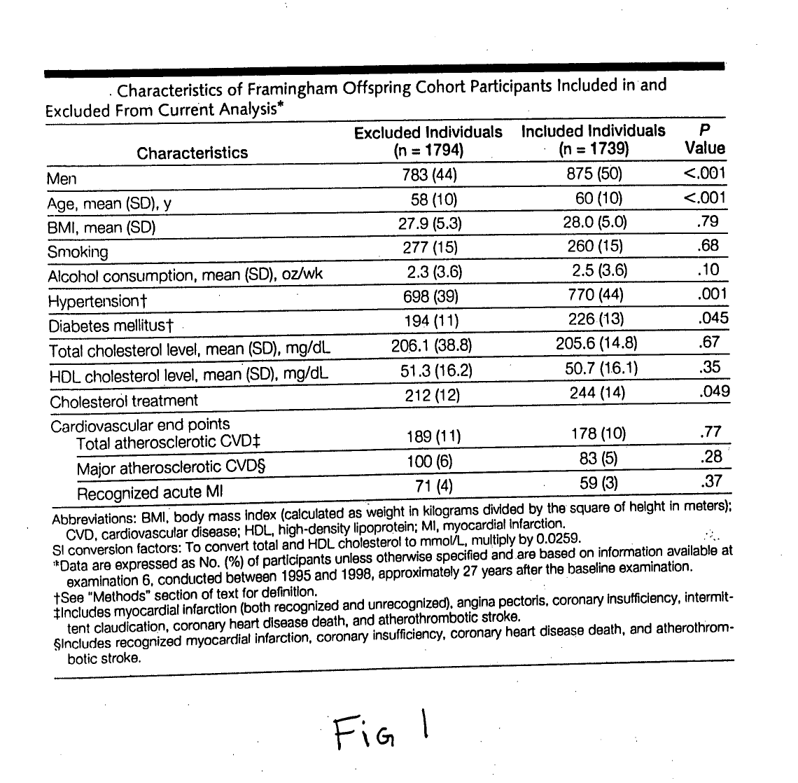

Subjects included in the current study are from a subset of unrelated individuals (N=1739: 875 men and 864 women) from the Framingham offspring cohort participants who provided blood samples for DNA extraction at the sixth examination cycle (N=2933). Eligible subjects had to be unrelated (i.e., one person randomly selected from each extended family or biologically unrelated to any other Framingham participant); and selection was designed to include equal numbers of men and women. These criteria were established to provide a panel of DNA samples that would be suitable for multiple studies. Genotyping was carried out in 1811 subject DNA samples; 1739 of these had ESR]c.454-397T>C genotypes. Data for these 1739 individuals are used throughout the current study. According to data from the sixth examination cycle (FIG. 1), the 1739 selected subjects were similar to the other Framingham offspring cohort members with respect to the prevalence of CVD events and risk factors, except for a larger percentage of men (50% vs 44%; P<0.001), slightly older (mean age: 60 years vs. 58 years; p=0.0001), higher prevalence of hypertension (44% vs. 39%; p=0.001), higher likelihood of diabetes mellitus (13% vs 11%; P=0.045), and higher likelihood of taking cholestrol-lowering medications (14% vs 12%; P=0.049).

Follow-Up and Data Collection

At each visit to the Framingham Heart Study clinic, offspring cohort participants underwent extensive evaluations. Examining physicians measured the seated blood pressure twice with a mercury column sphygmomanometer. The two blood pressure measurements were averaged to derive the systolic and diastolic pressures for that examination. Hypertension was defined as systolic blood pressure 140 mm Hg or more, or diastolic blood pressure 90 mm Hg or more, or current use of medication for treatment for hypertension. Diabetes was diagnosed as a fasting blood glucose level exceeding 125 mg/dL (6.9 mmol/L), or current use of medication for treatment of diabetes. Total and high-density lipoprotein (HDL) cholesterol were measured by a Centers for Disease Control and Prevention standardized laboratory. Participants were categorized as smokers if they currently smoked cigarettes or if they had quit within one year prior to the clinic visit. Body mass index was calculated as the weight in kilograms divided by the square of the height in meters (kg/m²).

At each clinic visit, a CVD history was obtained and hospitalization records were collected routinely for subjects with suspected interim CVD events. For subjects who did not attend a clinic examination a health history update was obtained by telephone and records from interim hospitalizations were obtained and reviewed. Prior to genotyping participant DNA samples, a panel of three physicians established the diagnosis of cardiovascular disease endpoints after reviewing all available medical records. A diagnosis of recognized acute myocardial infarction required simultaneous presence of at least two of the following three criteria: symptoms consistent with myocardial infarction, electrocardiographic changes of myocardial infarction, and diagnostic elevation of biomarkers. A diagnosis of unrecognized myocardial infarction was made when an electrocardiogram revealed new pathological Q waves in comparison with the last available tracing, and myocardial infarction was not known to have occurred in the interim. Angina was defined as the occurrence of typical ischemic chest discomfort of brief duration that was precipitated by exertion and relieved by rest or nitroglycerine. Coronary insufficiency was diagnosed when prolonged ischemic chest discomfort prompted a medical evaluation that included electrocardiographic evidence of transient ischemic abnormalities and there were no elevations in biomarkers indicative of infarction. Intermittent claudication was diagnosed when a participant reported recurrent lower extremity discomfort characteristic of ischemia occurring with exertion and relieved with rest. Atherothrombotic stroke was diagnosed when an unequivocal neurologic deficit lasting more than 24 hours occurred and an embolic cause was not found. In more than 90% of strokes, magnetic resonance or computerized tomographic imaging was available to aid in the diagnosis.

All subjects gave written informed consent (including that for DNA analysis) at each clinic visit and the examination protocol was approved by the Institutional Review Board at Boston Medical Center (Boston, Mass.).

DNA Extraction and Genotyping

Genomic DNA was extracted from peripheral blood leukocytes from offspring cohort participants attending their sixth examination cycle (1993-1998) using standard methods.

We used the current recommendations of the Human Genome Variation Society for the description of sequence variation and described the polymorphism in relation to a specific human ESR1 cDNA sequence (Accession number NM_{—}000125) where position 454 of the coding sequence was the first nucleotide of the start of the next closest exon to the marker we term ESR1 c.454-397T>C, and the variation was 397 nucleotides (according to the current chromosome 6 reference genomic contig, NT_{—}023451, version 12) upstream in the intron. Three other three polymorphisms, one intronic and two in exons, were studied to determine whether any association identified was specific to ESR1 (Matsubara et al., Arterioscler. Thromb. Vasc. Biol. 17:3006-3012, 1997; Kunnas et al., BMJ. 321:273-274, 2000; Lu et al., Arterioscler. Thromb. Vasc. Biol. 22:817-823, 2002; Evangelopoulos et al., Clin. Chim. Acta. 331:37-44, 2003; Petrovic and Peterlin, Cardiology. 99:163-165, 2003; Lehtimaki, et al., J. Mol. Med. 80:176-180, 2002; Herrington et al., N. Engl. J. Med. 346:967-974, 2002; Herrington et al., Circulation. 105:1879-1882, 2002; Mizunuma et al., Bone. 21:379-383, 1997; Becherini et al., Hum. Mol. Genet. 9:2043-2050,2000; Langdahl et al., J. Bone Miner. Res. 15:2222-2230, 2000; Kang et al., Cancer Lett. 178:175-180, 2002). These polymorphisms were named in a similar fashion to ESR1 c.454-397T>C.

The ESR1 c.454-397T>C and ESR1 c.454-351A>G single nucleotide polymorphisms were detected by polymerase chain reaction amplification and PvuII or XbaI restriction fragment length analysis (Castagnoli et al., Nucleic Acids Res. 15:866, 1987; Yaich et al., Cancer Res. 52:77-83, 1992). Genotyping was blinded to participant characteristics and allele calling was carried out independently by two separate investigators. The T allele of ESR1 c.454-397T>C codes for presence of the PvuII site and has been termed the p allele in some previous reports; likewise the C allele, which codes for absence of the site, has been termed the P allele. The A allele of ESR1 c.454-351A>G codes for presence of the XbaI site and has been termed the x allele in some previous reports; likewise the G allele, which codes for absence of the site, has been termed the X allele.

ESR1 c.30T>C (rs2077647) genotype was detected by a TaqMan™ assay. Nucleic acids were amplified under the following conditions: 95° C. for 10 minutes; 40×: 95° C. for 15 seconds, 62° C. for 1 minute, 72° C. for 1 minute (5′-3′ probe sequences: VIC-ACCAAAGCATCTGGGATGGCCC-TAMRA (SEQ ID NO:1), 6FAM-CCAAAGCATCCGGGATGGCC-TAMRA (SEQ ID NO:2), 5′-3′ primer sequences: GACCATGACCCTCCACACCCTGGATCTGATGCAGTAGG (SEQ ID NO:3)).

For each ESR1 c.975C>G (rs1801132) allele, a 17-mer oligonucleotide centered on the polymorphism was synthesized (5′-3′ oligo sequences: GAGCCCCCCATACTCTAGAGCCCCCGATACTCTA (SEQ ID NO:4)), end-labeled with [γ³P]ATP by using polynucleotide kinase (New England Biolabs, Beverly, Mass.), and hybridized at 52° C. in a tetramethyl ammonium chloride (TMAC)-based buffer to Hybond™ N+membranes (Amersham Pharmacia Biotech, Buckinghamshire, England) supporting PCR products. PCR conditions: 95° C. for 5 min; 35×: 95° C. for 30 s, 54° C. for 30 s 72° C. for 1 min; 72° C. for 10 min. (5′ to 3′ primer sequences:

| | |
|---|---|
| TTCACCTGTGTTTTCAGGGAGCTGCGCTTCGCATTC | (SEQ ID NO:5)) |
| TTAC. | |

Statistical Analysis

The observed genotype frequencies were compared, using a chi-square test, to determine if they were in Hardy-Weinberg equilibrium.

Although enrollment of participants in the offspring cohort of the Framingham Heart Study began in 1971, we were only able to study individuals who survived until the sixth examination at which blood samples were taken for DNA extraction (27 years after study enrollment). Thus, to reduce bias, we used a case control study design, presenting figures for prevalence rather than incidence of cardiovascular disease.

Association analyses examined the relation of c.454-397T>C genotype to prevalence of atherothrombotic CVD events. Three CVD endpoints were studied: (1) total atherosclerotic CVD events, defined as: (MI [both recognized and unrecognized], angina pectoris, coronary insufficiency, intermittent claudication, coronary heart disease death, or atherothrombotic stroke), (2) major atherosclerotic CVD (recognized acute MI, atherothrombotic stroke, coronary insufficiency, or coronary heart disease death), and (3) recognized acute myocardial infarction.

Of the 1739 men and women, initial qualifying events for total atherosclerotic CVD included 52 MIs (both recognized and unrecognized), 75 cases of angina pectoris, 8 cases of coronary insufficiency, 31 with intermittent claudication, 0 CHD deaths, and 14 atherothrombotic strokes. Initial major atherosclerotic CVD included 57 MIs, 10 cases of coronary insufficiency, 0 CHD deaths, and 16 atherothrombotic strokes. There were 59 cases of recognized MI (including those qualifying as initial events for the other two outcome categories as well as subsequent events) in analyses restricted to that endpoint.

Multivariate logistic regression was carried out with adjustment for age and sex and additionally for CVD risk factors measured at the first examination cycle (body mass index, hypertension, diabetes mellitus, total cholesterol, HDL-cholesterol, and cigarette smoking). Correction was made for overestimation of odds ratios in common events (Zhang and Yu, JAMA 280:1690-1691, 1998). The nominal threshold for significance was set at p=0.05 for comparison of baseline traits and p=0.01 for association analyses. Statistical analyses were performed using the SAS system (SAS Institute, Inc., Cary, N.C.).

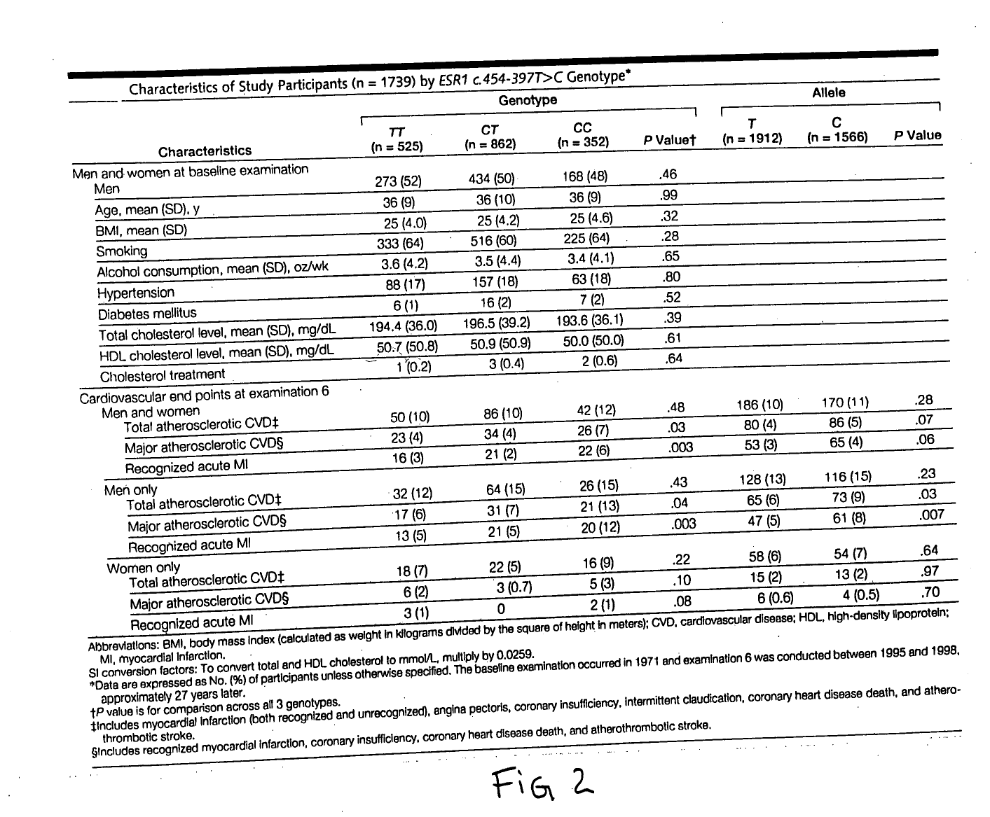

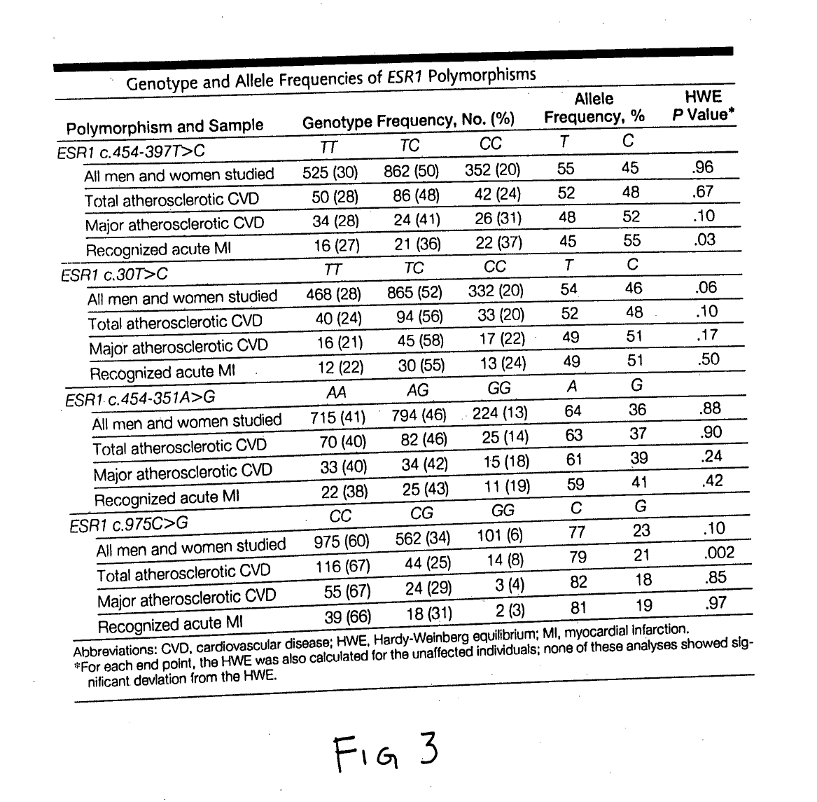

Results: The characteristics of the 1739 unrelated study subjects, at the baseline examination cycle of the Framingham Heart Study offspring cohort are shown in FIG. 2. The distribution of ESR1 c.454-397T>C genotypes was in Hardy-Weinberg equilibrium (FIG. 3) (P>0.05) except in the group of individuals with recognized myocardial infarction (P=0.03).

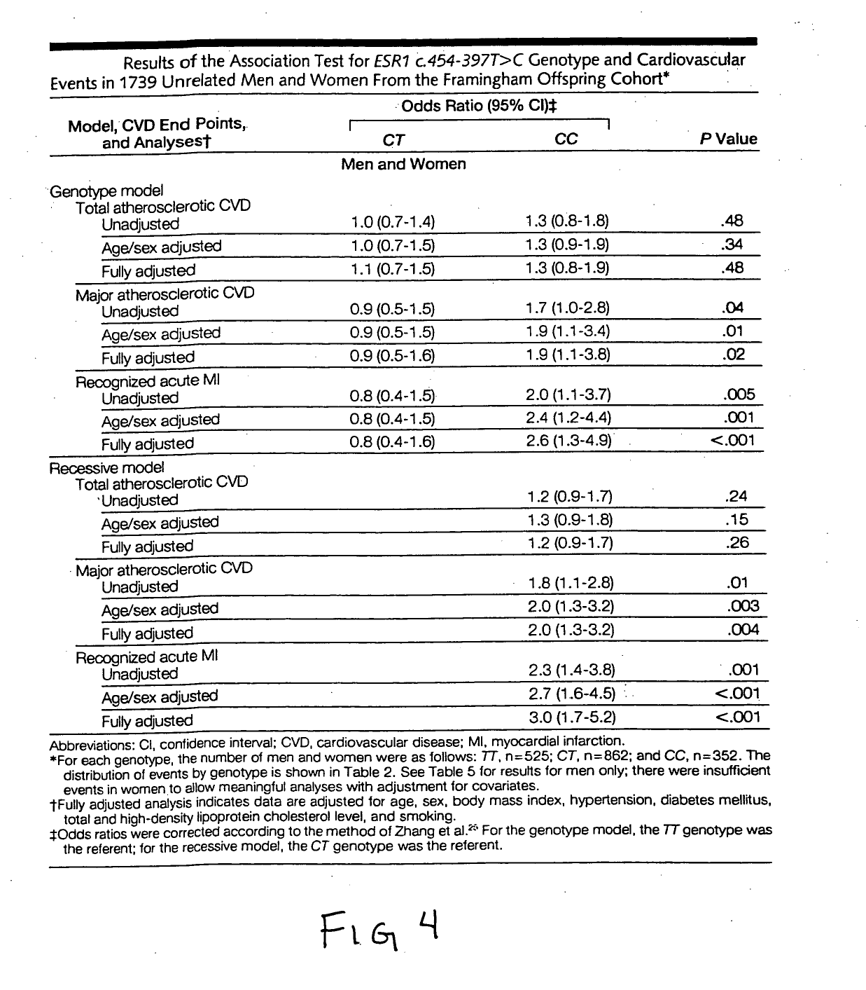

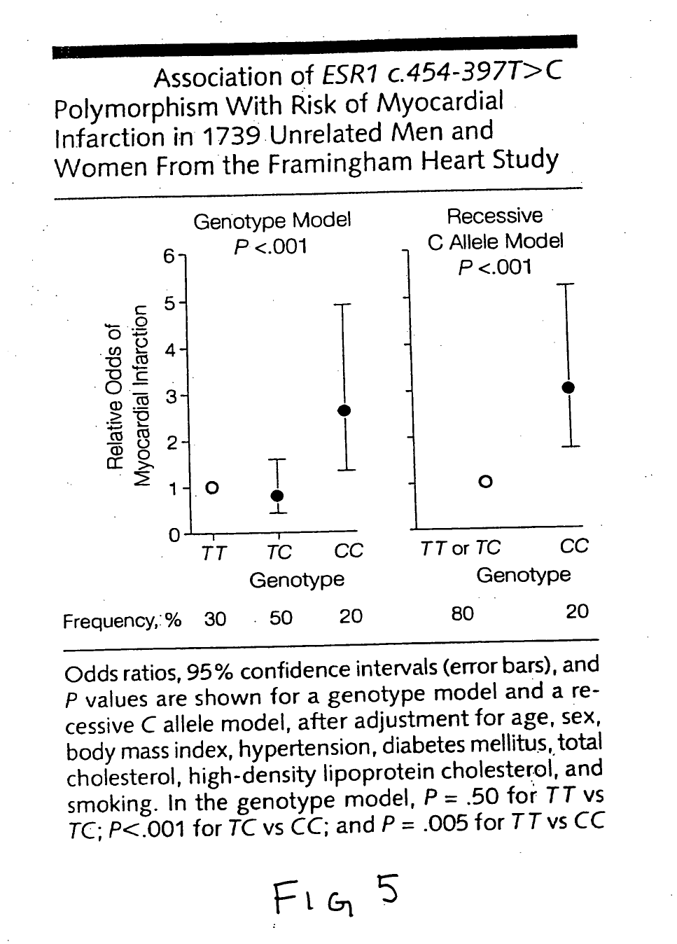

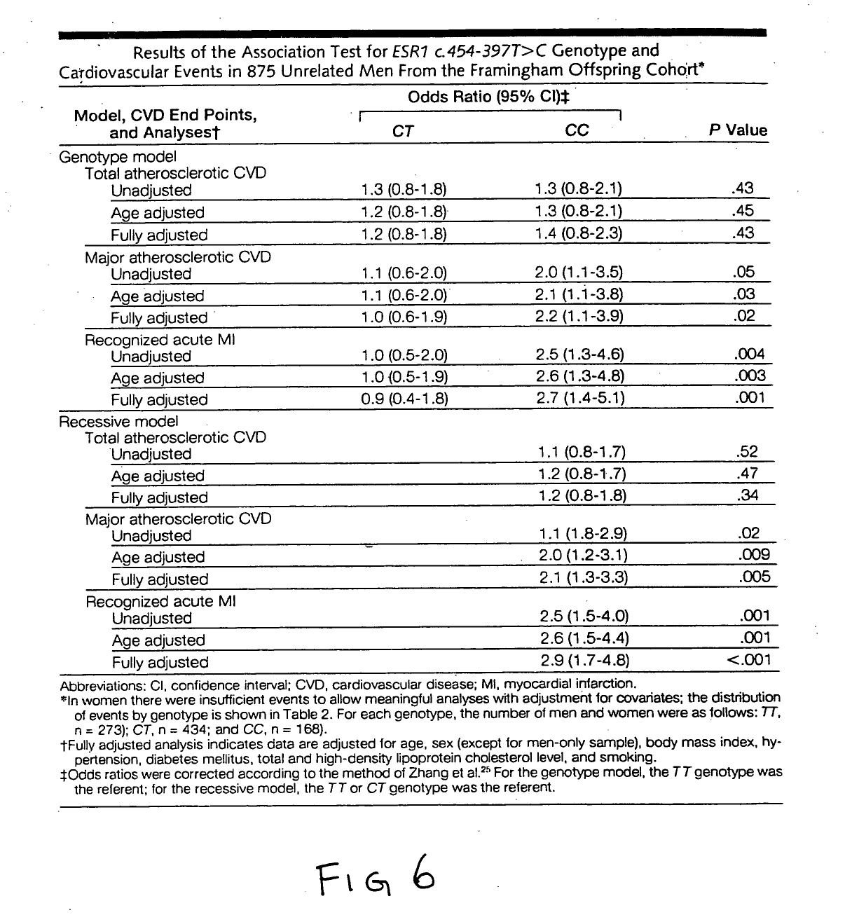

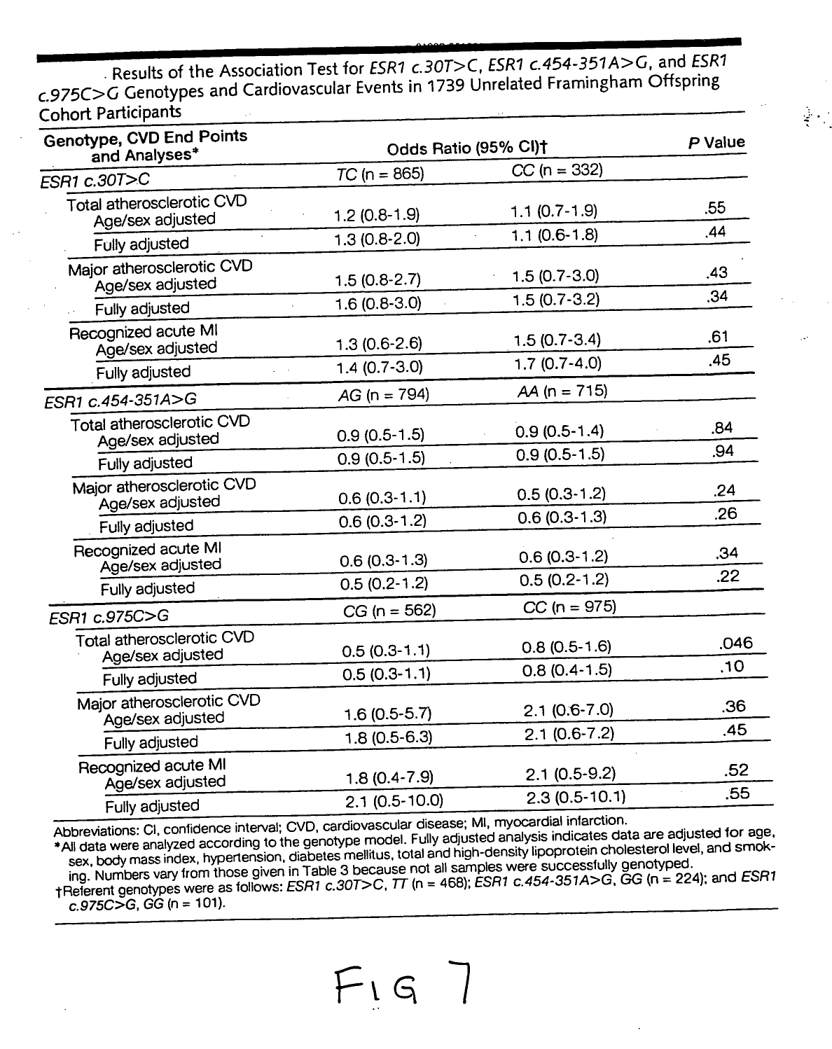

The results from analyses treating each genotype as a distinct group (genotype model) suggested a recessive effect of the C allele (FIGS. 4 and 5); when analyses were computed for the recessive model, the level of statistical significance increased for both major atherosclerotic cardiovascular events and recognized myocardial infarction (FIGS. 4 and 5). Subjects homozygous for the C allele comprised 20% of the study sample and compared with subjects carrying either one or two copies of the T allele, they had substantially higher odds of major atherosclerotic CVD and recognized myocardial infarction (FIG. 4). For individuals with the CC genotype, a recessive C model yielded an odds ratio [OR] of 2.0 (95% confidence interval [CI] 1.3-3.2; P=0.004) for major atherosclerotic CVD and 3.0 (95% CI 1.7-5.2; p=0.0001) for recognized MI. Survival analysis was performed for comparison. The results were very similar. For example, after full adjustment and under a recessive model, association of MI with the ESR1 c.454-397T>C polymorphism gave a hazard ratio of 3.0 (95% CI 1.7-5.1) with P=0.001 in men and women combined, almost identical to the odds ratio reported in FIG. 4 and discussed herein. The deviation from Hardy-Weinberg equilibrium within the individuals with recognized MI (FIG. 3) is consistent with the association being genuine; 37% of individuals with recognized MI and only 20% of the general study population had a CC genotype. When we limited the regression analyses to men the results remained significant (FIG. 6). Small numbers of events among women precluded studying them separately. In contrast, no differences in total atherosclerotic CVD by genotype were observed. For comparison we subsequently extended our investigation to three other highly informative and commonly studied single nucleotide polymorphisms in ESR1: c.454-351A>G in intron 1 (also called IVSI-354 A/G, or the XbaI restriction site), c.30T>C in exon 1 (T30C, Ser10Ser, a silent change) and c.975C>G (C1335G, +975C/G, Pro325Pro) in exon 4. The pairwise linkage disequilibrium coefficients (D′) between ESR1 c.454-397T>C and the three other polymorphisms: ESR1 c.454-351A>G, ESR1 c.30T>C, and ESR1 c.975C>G are respectively 0.992, 0.823, and 0.362. The genotype frequencies of the four markers were similar to those reported previously (Herrington et al., N. Engl. J. Med. 346:967-974, 2002; Willing et al., J. Bone Miner. Res. 13:695-705, 1998; Deng et al., Osteoporos. Int. 9:499-507, 1999). With the exception of ESR1 c.975 C>G among the total atherosclerotic CVD group the distribution of genotypes was in Hardy-Weinberg equilibrium for all three SNPs and results for association with all three endpoints were negative after adjustment for covariates (FIG. 7).

The CC genotype of ESR1 c.454-397 was present in one-fifth of our population-based sample and it was associated with a three-fold increase in odds of MI. These findings underscore a potentially important role of ESR1 in influencing the development of atherosclerosis and/or in accelerating the transition from subclinical atherosclerosis to plaque rupture and acute thrombotic CVD events such as MI and stroke. The frequency of the CC genotype was 20% in the whole study sample, but it was 24% in the individuals with the broadest definition of atherosclerotic CVD, 31% in the individuals with major atherosclerotic CVD and 37% in the individuals with recognized myocardial infarction. For the later two endpoints, there was statistically significant evidence of association.

Although several investigations of the ESR1 c.454-397T>C variant and cardiovascular risk factors have been reported, only one, a postmortem analysis found a significant association of this variant with coronary artery disease (Lehtimaki et al., J. Mol. Med. 80:176-180, 2002). In that study of 300 white Finnish male autopsy cases, between 33 and 69 years of age, from the Helsinki Sudden Death Study, the ESR1 c.454-397T>C variant was significantly associated with coronary artery disease in the subset of men of 53 years of age or older. In this group of 142 men, 69 had coronary thromboses and there was a significant 10.2 fold (P=0.04, 95% CI, 1.1-103.5) higher frequency of coronary thrombosis among the CC individuals when compared to the TT individuals. The heterozygotes had an intermediate risk that was not significantly different from that of the TT homozygotes. In addition, the CC cases had on average a 5-fold greater area of complicated lesions than the TT cases (age adjusted P=0.001) (Lehtimaki et al., J Mol Med. 80:176-180, 2002). There may be some inter-population heterogeneity of environmental or genetic factors that resulted in our study finding no difference in CVD susceptibility in heterozygotes when compared to the TT homozygotes.

The design and results of our study include many of the features that are considered desirable components of the ideal association study, such as a large sample size, small P values, an association that makes biological sense, and alleles that may affect the gene product in a way that is of potential physiological relevance.

There are many reports of the ESR1 c.454-397T>C variant, mostly in bone mineral density or cancer studies. We observed a C allele frequency of 45% and other reports of US Caucasians provided very similar figures (45-49%) (Herrington et al., N. Engl. J. Med. 346:967-974, 2002; Willing et al., J. Bone Miner. Res. 13:695-705, 1998; Deng et al., Osteoporos Int. 9:499-507, 1999), while studies of other ethnicities provided a wider range of results (3 1% Holmberg-Marttila et al., Calcif. Tissue Int. 66:184-189, 2000; Kim et al., Menopause. 8:222-228, 2001; Lau et al., Bone. 29:96-98, 2001), to 55% in the SNP database (see http://www.ncbi.nlm.nih.gov/SNP/).

There are several biologically plausible explanations for the mechanism by which ESR1 variants influence the risk of MI (Mendelsohn and Karas, N. Engl. J. Med. 340:1801-1811, 1999). For example, a number of hormone-sensitive alterations in hemostatic and/or fibrinolytic variables may be involved (Mendelsohn and Karas, N. Engl. J. Med. 340:1801-1811, 1999; Vandenbroucke et al., Lancet. 344:1453-1457, 1994; Braunstein et al., Chest. 121:906-920, 2002; Prandoni et al., N. Engl. J. Med. 348:1435-1441, 2003). Estrogen receptors have an important role in normal vascular physiology that is not limited to women or other female mammals (Mendelsohn and Karas, N. Engl. J. Med. 340:1801-1811, 1999; Brouchet et al., Circulation. 103:423428, 2001; Pare et al., Circ. Res. 90:1087-1092, 2002; Pendaries et al., Proc. Natl. Acad. Sci. USA 99:2205-2210, 2002; Zhu et al., Science. 295:505-508, 2002). ESR1 has been shown in basic and animal studies to mediate three direct effects of estrogen on the vessel wall. These include acceleration of reendothelialization, alteration of endothelial NO production, and inhibition of the vascular injury response. These animal studies also demonstrated the importance of estrogen receptors in cardiovascular physiology in both males and females. This is consistent with human studies that further support the importance of estrogen receptors in both sexes (Sudhir et al., Lancet. 349:1146-114, 1997; Sudhir et al., Circulation. 96:3774-3777, 1997).

In summary, our results reveal an association between a common estrogen receptor genotype, ESR1 c.454-397CC, and increased odds of MI. This association persists after adjustment for traditional CVD risk factors and provides support for the importance of estrogen receptors in CVD susceptibility, especially in men. Estrogen receptor variation also has potential to explain recent data regarding the effects of combination hormone replacement therapy on CVD risk in women (Hulley et al., JAMA. 280:605-613, 1998; Rossouw et al., JAMA. 288:321-333, 2002).

### Example 2

### Association of the ESR1 c.454-397T>C Polymorphism with Atherothrombotic Stroke

From April 1989 to April 1994, 3012 healthy Caucasian men between the ages of 51 and 60 years, who were registered with nine primary care practices in the United Kingdom, were recruited for prospective surveillance (Miller et al., Thromb. Haemost. 73:82-86, 1995; Miller et al., Thromb. Haemost. 75:767-771, 1996). To be eligible for surveillance, the subjects had to be free from a history of unstable angina, MI or evidence of silent infarction, coronary surgery, aspirin or anticoagulant therapy, cardiovascular disease, malignancy (except skin cancer other than melanoma), or any condition precluding informed consent. Follow-up is ongoing, with a current median follow-up of 10.5 years. Only 26 individuals have been lost to follow-up. They were censored at the point they were last known to be alive and healthy and data collected from them has been included in the current analysis. Participants were asked to attend non-fasting and were instructed to avoid heavy meals and to refrain from smoking or vigorous exercise from the midnight preceding examination.

Participants answered a questionnaire concerning their previous medical history and smoking habit and were classified as “never smokers,” “ex-smokers” or “current smokers.” Men who had at some time smoked at least one cigarette per day for at least a year were defined either as current smokers or ex-smokers. To be an ex-smoker, a man had to have stopped smoking for at least one year. Even if they had reduced their smoking to less than one cigarette per day in the last year, they were still categorized as current smokers. All others were categorized as non-smokers. To be entered in the study, men had to have had a baseline ECG free of evidence of a previous MI. This evidence was the presence of a major pathological Q wave (Codes 1-1 or 1-2-1 to 1-2-7, or 1-2-8 plus T wave codes 5-1 or 5-2) on Minnesota criteria. This resulted in the exclusion of 42 men with changes indicative of MI. Weight (in kg), height (in m) and systolic blood pressure (SBP) measurements were recorded, and venous blood samples were collected for plasma and DNA analysis. Participants were recalled annually for interview and repeat plasma analysis. A routine ECG was repeated at the sixth examination. CHD endpoints, recorded to June 2004, were: 45 fatal MIs, 102 non-fatal MIs, 19 silent MIs, and 39 coronary revascularization procedures. In the same period, there were 54 strokes.

Biochemical and Clinical Measurements, and Genotyping

A 5 ml sample of venous blood was taken using a Vacutainer™ (Becton Dickinson, Cowley, Oxford, UK) from each patient and transferred to plastic screw-cap vials, which were stored at −40° C. pending analysis. Diabetes status was self-reported. Hypertension was defined as a systolic blood pressure≧140 mmHg or a diastolic≧90 mmHg or use of anti-hypertensive medication. Cholesterol and triglyceride concentrations were measured using automated enzyme procedures.

Blood pressure was recorded twice per examination, with a random zero sphygmomanometer, after the subjects had been seated for at least 5 minutes, and the results were averaged for statistical analysis. Height (m) was measured on a stadiometer and weight (kg) on a balance scale to calculate BMI, kg/m².

Genotyping was carried out, with inclusion of positive and negative genotypic control samples on each plate, using standard methods. Genotype data from 2557 men was included in the current study.

Statistical Analysis

Statistical analysis was conducted using Intercooled STATA version 8.2. Association of genotype with risk of Stroke was assessed using Cox's proportional hazards model, with significance assessed by the likelihood ratio test. Results are presented as Hazard Ratio (HR) with 95% CI. Results were adjusted for age and practice unless otherwise stated.

Stroke ESR1 Association Results: In our prospective study of 2,709 men we obtained significant association of ESR1 c.454-397T>C genotype with atherothrombotic stroke. The relative risk, adjusted for age and medical practice, was 1.92 (95% confidence interval 1.06-3.48) for individuals with the CC genotype at that polymorphism, when compared to individuals with CT or TT genotypes, P=0.03. Analyses that were additionally adjusted for smoking status, body mass index, cholesterol, triglyceride level. Blood pressure and diabetic status provided similar results.

### Example 3

### Association of the ESR1 c.454-397T>C Polymorphism with Myocardial Infarction-A Study in 7,072 Men

We aimed to determine more clearly whether the CC genotype at ESR1 c.454-397T>C is associated with increased odds of non-fatal MI among men. In this study, we included only cohorts of Caucasian men not recruited on the basis of CHD risk factors, for example, familial hypercholesterolemia, or familial premature CHD. We studied 4,025 men, drawn from the prospective population based Northwick Park Heart Study (NPHSII) in the UK (Cooper et al., Circulation 102:2816-2822, 2000) and two large case/control studies of MI, from Poland and the US, selected from the Global Repository at Genomics Collaborative (Ardlie et al., Am. J. Hum. Genet. 71:304-311, 2002). Here we present a meta-analysis of the results of these unpublished studies, along with published reports, from the Framingham (Lehtimaki et al., J. Mol. Med., 80:176-180, 2002) and Rotterdam Studies (Schuit et al., JAMA., 291:2969-2677, 2004), of a total of 7,072 men with detailed covariate information, including 768 men with acute, non-fatal MI (both fatal and silent events were excluded). Meta-analysis was performed in Stata 8.2 (Stata Corporation, Texas) using the user-written command ‘meta’. This uses inverse-variance weighting to calculate fixed and random-effects summary estimates.

We also carried out analysis of ischemic hear disease (IHD), defined in NPHSII and Rotterdam using international classification of disease (ICD) definitions: acute MI, IHD death, or revascularization procedures (percutaneous transluminal coronary angioplasty or coronary artery bypass surgery). In Framingham, IHD was defined as recognized MI, coronary heart disease death, angina pectoris, or coronary insufficiency (revascularization procedure data were not available).

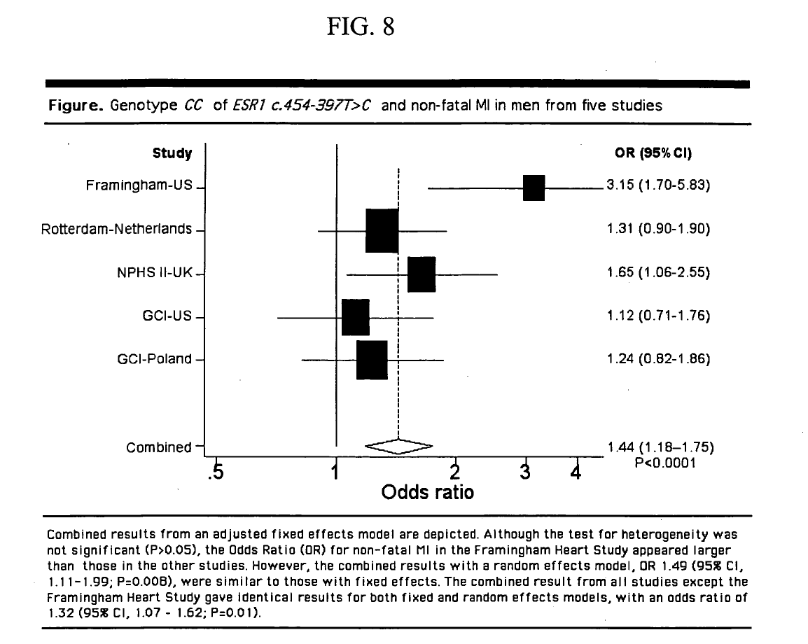

Comparing men with the ESR1 c.454-397CC genotype to those with CT or TT genotypes, a covariate adjusted fixed effects model, gave a pooled odds ratio of 1.44 (95% CI, 1.18-1.75; P<0.0001) for non-fatal MI (FIG. 8). The pooled odds ratio for IHD in the three prospective studies was 1.24 (95% CI, 1.02-1.53, P=0.03). Almost identical results were obtained from analyses that were unadjusted, or adjusted for age, body mass index, plasma cholesterol level, hypertension, diabetes and smoking status.

Sixty-two percent of the weight of our model for MI is derived from previously unstudied cohorts, making it unlikely that our results have been affected by publication bias. Our findings also show consistency across five Caucasian cohorts from four countries. Estrogen receptors are required for normal vascular physiology in males (Mendelsohn, Circ. Res., 93:1142-1145, 2003), however, the mechanisms that underlie the association are not clear at present, though several studies provide evidence that the ESR1 c.454-397C allele results in a relatively high level of ESR1 transcription. Our study cohorts contained only 45 previously unpublished fatal MIs so had insufficient power to resolve whether there is a similar, larger (Lehtimaki et al., J. Mol. Med., 80:176-180, 2002) or opposite (Schuit et al., JAMA., 291:2969-2677, 2004) result for fatal versus non-fatal events.

Study of 7,072 Caucasian men from four countries, provides evidence that the common ESR1 c.454-397CC genotype, present in roughly 20% of individuals, is a risk factor for non-fatal acute MI, and IHD, after adjustment for established cardiovascular risk factors. For MI events in the studied population the high risk genotype confers a population attributable risk of 6.7%, corresponding each year in the US to over 25,000 MIs, and resulting in billions of dollars in lost productivity, hospital and medical expenses.

A number of embodiments of the invention have been described. Nevertheless, it will be understood that various modifications may be made without departing from the spirit and scope of the invention. Accordingly, other embodiments are within the scope of the following claims.

TABLE 1

Genomic sequence containing the c.454-397T > C polymorphism. This
sequence is derived from GenBank ® NT_025741, GI:37551196:nucleotides
56316184-56320184 (Homo sapiens chromosome 6 genomic contig). The site
of the T > C polymorphism is shown as a bold, underlined T in row 29
of the sequence.

| | |
|---|---|
| ACCTCCAAAGGCACTAAGCTTCTACAGTTAGATATTCATAGCTGCTTCCTACTGACTTGAATCATGCATA | (SEQ ID NO:6) |
| GGATATTAGTAAACAAGCAATAAAAAGATTTGAGGTTGATGGGGGTGGGTTCAACAGCATGGTGGTGAAA | |
| TGGAAAGAGATGGGTAACAGAATATGAACTAGAATTGAAAACTGTGAGCCAGTGCTCTCTAATGAACATT | |
| AAAAAATAAAGAATTCCTATTTGAGGCTGCCAACCTCAGAACTAAGTTATTTAGAATGGACGAAATTGGC | |
| AAAGTCAGACGTACTCAACCCAAGGAGCCAATATTTTGTGAATATTATGGCAAATGTAGTTTGAGAACCA | |
| CTACCACAAAATTGTGAACCATAATAATGACTGAGAAGGCAGGGAGAGGTTATACAATTTGGGCTAAAAG | |
| GAAAGACAGGGCTTGTGAAGGGGAGCGCCAGTGAAAGTCAGTGTGGTTCGGGTATTTGGGTGGGGACTGG | |
| AAGCAGGAAGCTTGAGCTTCCTTTGCCAAGAGACCCTGCTGGAAGGGCTATCATCAATTGACTTTAGCTC | |
| ATCTTAGGATTTTCATTTTTTAAAAAATGTTCACAGGAACCTTCACTCCATCTATACTTTCAATGTCTGC | |
| CTACCTTTCTTTCTTATACAACTTTGAACACTCTCTCCATTCATTTAAATATATTATGGAGTGCCAACTA | |
| CATGCCAGGTACTGTGCTGGGCTCTTATTCCACCTTTATTTGATTGCACATGCCTGCCAAGTCCTGGGCC | |
| AATATAACATCTACTCCTATGTCTGGTCTGGCGAGAGATGCAAACTCATCTTCCTCTACTTTCCTTACCT | |
| CCTTCCTTCCAGTCTTCTTCAAGTTGTCTTCATTGAGGCAATTTCTTTTACCTGTGTTTTTAATCCCAAC | |
| TCCTCTAGTTTCCTTCTTGGCTTTATTCTTTTATCTTCCTCTTTGTGCTTTCAAACATTCCCTTTCTCCT | |
| GGCCCATGCCCTTCAGTCTACACGAGGCCTTCTCAAGTCTCTTCATTCTAAAAAATTCATTTTCTTGGGT | |
| CTTATATTCTTCAGCTGCCACCCTATCTGTATCTTTTCCTATTCTCCTCCAAGTTCTCAAAGGAATGCCT | |
| TCCCTCATTTTCATCTCCTTACATTCCATCTGCTGAATTTTGGCTTGTGCCTGTACCTGTCTAAGGAAAC | |
| TCCTTGCTAAGAGTCTGCTTTGTCAGGTCTGAATTCACTTAACCAGTCTTTGCTTTGTTGGACTTCTCTG | |
| CCCCATTTGCCATTCTTGATCATCCTCTCCATAAACCTTTCTACTTAAAGCATTTTACTTCCTTATTTTC | |
| TTGGTTTTCCTAGAATCTCCTTACTGTTCATTTTCAGCTTCCTTTCTGTGTTCCTCTTCTCTTCCTACAT | |
| TTTTTTTTAGCTTTCTACTTTCTTAAAGCATTTTACTTCCTTATTTTCTTGGTTTTCCTAGAATTTTCTT | |
| ACTGTTCATTTTCAGTTTCCTTTCTGTGTTCCTCTGATTGTCTCTCTTTCTACATTTTTTTTTTCTGTGT | |
| TCCTCTGATTTTCACGCAGTCTGGAGTTGTCATGATCAATCATAGCCTACTGCAGCCTCGACATCCTAGG | |
| CTCAAGTGATTCTCCCACCTCAGCCTTACAAGTAGCTAGGACTACAGTCACACATCACCATTCTCAGCTA | |
| ATTTTTTTAAGAAGCATTTTTATAGAGATGGAGTCTTGCTATATTGTGCAGGCTGGGCTCAAACTACAGG | |
| GCTTAAACAATTCTCCTGCTTTGGCCTCCCAAAGTGCTGGGATTCCAGGCATGAACCACCATGCTCAGTC | |
| TCTACATGTTCCTAAAGAGGAGTTTTGAATATTGAAGAACAGTATTTTCAAATTACATTATTCAAGTTAT | |
| AAAAACTGATATCCAGGGTTATGTGGCAATGACGTAAAAATTTGAATTGTTATTTTTTTGACACATGTTC | |
| TGTGTTGTCCATCAGTTCATCTGAGTTCCAAATGTCCCAGCTGTTTTATGCTTTGTCTCTGTTTCCCAGA | |
| GACCCTGAGTGTGGTCTAGAGTTGGGATGAGCATTGGTCTCTAATGGTTCTGAAATAATTGTATATTCCT | |
| GCAAAAACATTAAGTCTATTAGAAACCAGCTAATTTCATTTTGTCATTTTTATAGGTAACATATTCTGGT | |
| GCAGGTAGTATGTTTTTAAAACAAGTTTGCAATAAACAATTTCCCCTCAAGGTTAATATAATAGGCAACA | |
| CCTTTTGCTGCAACAGACGGCAAGAGGTAATGAAAGATTAGCTTACATTATGATTCATTATTTCAAAATG | |
| TCAGGATAAAGTGGATCTGCTGCATCTCCCAGAGAGTGCATGTTTTGCTTTTCTAATGTTAATGGATTTA | |
| CTGTTTTTTTCCCCCCAGGCCAAATTCAGATAATCGACGCCAGGGTGGCAGAGAAAGATTGGCCAGTACC | |
| AATGACAAGGGAAGTATGGCTATGGAATCTGCCAAGGAGACTCGCTACTGTGCAGTGTGCAATGACTATG | |
| CTTCAGGCTACCATTATGGAGTCTGGTCCTGTGAGGGCTGCAAGGCCTTCTTCAAGAGAAGTATTCAAGG | |
| TAATAGTGTGTTGAAAACGACTTCTATTTTTGATCCTATGAGCAGATCCTAAGAGCCAAAGCGACTGAGG | |
| AAGGAAGACATAGAATCAGCCATTTGTACAAAACATGAATCCCTAGTAGGTCCACTAGTATCTTTGGTAG | |
| AAACATGGAGAAGAGACAGGATCTCAGGAGAAGGAGTTGACACATGGCAGGGCAGCTGAGGCTGAGTAAT | |
| TCCGCTTCCTTCCTTTGGCAAGACTCAATCAGTCTTGAGCAACTCTACAGAAGAATTCCACTAGCTGGAT | |
| CTCTGAGGAAAAAAGAAATGTTGTCTGTGCCCTGACTGGGCAATGCCAGATGGACATTCATGTTTGGTAG | |
| GCAACTTTGCCTATATGATCTGGTATATGCTGTTAATTGTCCATGCATAATTATCTCTCTACTCAGGCCT | |
| TGTCCAGGCAAATATTCTGTTTTGTTCTAGTTTAGCTTCTTCTCCCCTTTCTCTCTTCCATCTCTTTCTT | |
| GTCTCAATGGATGACAGGATATTTTGCTATGAGCTGACTCAGTGGTTGGTGTCTTGTAATGGGGAGATAT | |
| CATCTTTATCAAACAGTTATTAAGTATCTACCTGTAGCATTTCATTTTCCCGCCTGCCTCCATTGTTTTC | |
| TTGTCTATAGTTTGCCAATTATAGCTAATATACGGAGAGCTATACTTTATTTCTACTCCAGAAATGTCTC | |
| TATTATTGCATTATAATAGGATACCCTGGGGAAACACTAATCATTTTTACTACCTAAAATACCTATGCTG | |
| AATATCCTTTATCTGATAGGAACAGAGATCTGACAGCAGCTTAGGCTAACCAAATTCATTTTTTATCTTA | |
| AGTGTGGGGCATTTTTCTCTCTTCTTATTCTTTACCTTTTCAGCTTAAGTGAAGGTTAGTATAAACACTA | |
| AGAATATTTCTGATGGAGTTTTCATGTGATTCCTTCTACAAAAACCCAGATTTAAGTAACTTGTTGAAAA | |
| CCAGAGTCCGCTAAGTTAATAAACACTGATTGAAGAAGTGATTCTCATGGACTTTCTGTGATAGCTCTTT | |
| CCTGCCCTGATATGAGATGAAAGCTGGGGGATGGTATATAGTATTTATTTTTCCTTCCCTTGCCAGTGGG | |
| ACTTTTTTTTTTTTTTTAAAAGCTGTTCATATCTTAATCGAGTAGCATGTGAGGTCAACATGGTCTATTT | |
| TAAAAGCATTTTCTTCGACACATTGCTTTTAACATCTTTTAGAACTCTGCTGTGAGACACATGGACTTTT | |
| TTGTTGGTATTTTTATACAATTAATGATATTCTCAATAGTAATCTTTGTGTGTGTATATATATAGAAATA | |
| AATTCTAAATGTAAGTTAATATATTTATTATTTTTCTAAACATATATAAATATATATATGCACACAGGCT | |
| ATTTAATTTTA | |

1. A method of evaluating a patient's risk for cardiovascular disease, the method comprising determining the genotype of the c.454-397T>C polymorphism of an estrogen receptor alpha (ESR1) gene of a patient, wherein the presence of a C allele in the c.454-397T>C genotype indicates that the patient has an increased risk for a cardiovascular disease.

2. The method of claim 1, wherein the method comprises determining the genotype in a nucleic acid which is derived from a nucleic acid sample obtained from the patient.

3. The method of claim 1, wherein the cardiovascular disease is manifest as atherosclerosis, acute myocardial infarction, angina pectoris, venous thrombosis, coronary insufficiency, coronary heart disease death, atherothrombotic stroke, or intermittent claudication.

4. The method of claim 1, wherein the patient is younger than 30 years of age.

5. The method of claim 1, wherein determining the genotype comprises exposing the nucleic acid to a restriction endonuclease that cleaves the nucleic acid at a Pvu II site under conditions and for a time sufficient to allow the endonuclease to cleave the nucleic acid.

6. The method of claim 5, wherein the nucleic acid has been amplified.

7. The method of claim 1, wherein determining the genotype comprises sequencing the nucleic acid.

8. The method of claim 1, wherein determining the genotype comprises restriction fragment length polymorphism analysis, allele specific oligonucleotide analysis, denaturing/temperature gradient gel electrophoresis, single-strand conformation polymorphism analysis or dideoxy fingerprinting.

9. The method of claim 1, further comprising determining whether the patient: (a) regularly smokes cigarettes or uses another tobacco product; (b) regularly exercises; (c) has high blood pressure; (d) has elevated blood cholesterol levels; (e) has genetic relatives who have cardiovascular disease; or (f) is experiencing a sign or symptom of a cardiovascular disease.

10. A method of predicting how a patient will respond to an anti-atherosclerotic therapy, the method comprising: determining the genotype of the c.454-397T>C polymorphism of ESR1 in a nucleic acid sample obtained from the patient, wherein the presence of a C allele in the c.454-397T>C genotype indicates that the patient is likely to benefit from therapy with an anti-atherosclerotic agent.

11. The method of claim 9, wherein the anti-atherosclerotic therapy is a lipid-lowering therapy.

12. The method of claim 9, wherein the anti-atherosclerotic therapy is an anti-platelet therapy.

13. The method of claim 9, wherein the anti-atherosclerotic therapy is an anti-coagulant therapy.

14. A method of determining whether a patient is likely to experience adverse side effects if subjected to a hormone-based therapy, the method comprising: determining the genotype of the c.454-397T>C polymorphism of ESR1 in a nucleic acid sample obtained from the patient, wherein the presence of a C allele in the c.454-397T>C genotype indicates that the patient has an increased risk of experiencing an adverse side effect associated with a hormone-based therapy.

15. The method of claim 14, wherein the therapy is a hormone replacement therapy.

16. The method of claim 14, wherein the therapy comprises the administration of an estrogen for the purpose of contraception.

17. The method of claim 14, wherein the adverse side effect is a sign or symptom of cardiovascular disease.

18. A computer-readable database comprising a plurality of records, each record comprising: (a) a first field comprising information reflecting the genotype of one or both alleles of a c.454-397T>C polymorphism of the ESR1 gene of a human subject, and (b) a second field comprising information concerning a cardiovascular parameter of the subject.

19. The database of claim 18, wherein the subject is between 20 and 60 years of age.

20. The database of claim 18, wherein the information contained with the first field is obtained before obtaining the information contained within the second field.

21. The database of claim 18, further comprising a field comparing the cardiovascular parameter to a clinical outcome associated with the parameter.

22. The database of claim 18, wherein the subject exhibits a sign or symptom associated with a cardiovascular disease.

23. The database of claim 18, wherein the subject has had a myocardial infarction or stroke.

24. The database of claim 18, wherein the cardiovascular parameter is high blood pressure, a high blood cholesterol level, an abnormal electrographic profile, or angina.

25. The database of claim 18, further comprising information reflecting the genotype of one or more additional nucleotides of the ESR1 gene, wherein the information about the genotype of the additional nucleotide(s) is associated with the information about the genotype of the c.454-397T>C polymorphism and the cardiovascular parameter.

26. The database of claim 18, wherein the second field comprises information concerning two or more cardiovascular parameters.

27. The database of claim 18, wherein the database comprises at least 50, 100, 250, 500, 1000, 1500, 1800, 2000, or 2500 records.


SEQUENCE LISTING (6 sequences)

SEQ ID NO: 1 - DNA, 22 bp, Artificial Sequence
  Probe sequence
accaaagcatctgggatggccc

SEQ ID NO: 2 - DNA, 20 bp, Artificial Sequence
  Probe sequence
ccaaagcatccgggatggcc

SEQ ID NO: 3 - DNA, 38 bp, Artificial Sequence
  Primer
gaccatgaccctccacaccctggatctgatgcagtagg

SEQ ID NO: 4 - DNA, 34 bp, Artificial Sequence
  Synthetically generated oligonucleotide
gagccccccatactctagagcccccgatactcta

SEQ ID NO: 5 - DNA, 40 bp, Artificial Sequence
  Primer
ttcacctgtgttttcagggagctgcgcttcgcattcttac

SEQ ID NO: 6 - DNA, 4001 bp, Homo sapiens
acctccaaaggcactaagcttctacagttagatattcatagctgcttcctactgacttgaatcatgcataggatattagtaaacaagcaataaaaagatttgaggttgatgggggtgggttcaacagcatggtggtgaaatggaaagagatgggtaacagaatatgaactagaattgaaaactgtgagccagtgctctctaatgaacattaaaaaataaagaattcctatttgaggctgccaacctcagaactaagttatttagaatggacgaaattggcaaagtcagacgtactcaacccaaggagccaatattttgtgaatattatggcaaatgtagtttgagaaccactaccacaaaattgtgaaccataataatgactgagaaggcagggagaggttatacaatttgggctaaaaggaaagacagggcttgtgaaggggagcgccagtgaaagtcagtgtggttcgggtatttgggtggggactggaagcaggaagcttgagcttcctttgccaagagaccctgctggaagggctatcatcaattgactttagctcatcttaggattttcattttttaaaaaatgttcacaggaaccttcactccatctatactttcaatgtctgcctacctttctttcttatacaactttgaacactctctccattcatttaaatatattatggagtgccaactacatgccaggtactgtgctgggctcttattccacctttatttgattgcacatgcctgccaagtcctgggccaatataacatctactcctatgtctggtctggcgagagatgcaaactcatcttcctctactttccttacctccttccttccagtcttcttcaagttgtcttcattgaggcaatttcttttacctgtgtttttaatcccaactcctctagtttccttcttggctttattcttttatcttcctctttgtgctttcaaacattccctttctcctggcccatgcccttcagtctacacgaggccttctcaagtctcttcattctaaaaaattcattttcttgggtcttatattcttcagctgccaccctatctgtatcttttcctattctcctccaagttctcaaaggaatgccttccctcattttcatctccttacattccatctgctgaattttggcttgtgcctgtacctgtctaaggaaactccttgctaagagtctgctttgtcaggtctgaattcacttaaccagtctttgctttgttggacttctctgccccatttgccattcttgatcatcctctccataaacctttctacttaaagcattttacttccttattttcttggttttcctagaatctccttactgttcattttcagcttcctttctgtgttcctcttctcttcctacatttttttttagctttctactttcttaaagcattttacttccttattttcttggttttcctagaattttcttactgttcattttcagtttcctttctgtgttcctctgattgtctctctttctacattttttttttctgtgttcctctgattttcacgcagtctggagttgtcatgatcaatcatagcctactgcagcctcgacatcctaggctcaagtgattctcccacctcagccttacaagtagctaggactacagtcacacatcaccattctcagctaatttttttaagaagcatttttatagagatggagtcttgctatattgtgcaggctgggctcaaactacagggcttaaacaattctcctgctttggcctcccaaagtgctgggattccaggcatgaaccaccatgctcagtctctacatgttcctaaagaggagttttgaatattgaagaacagtattttcaaattacattattcaagttataaaaactgatatccagggttatgtggcaatgacgtaaaaatttgaattgttatttttttgacacatgttctgtgttgtccatcagttcatctgagttccaaatgtcccagctgttttatgctttgtctctgtttcccagagaccctgagtgtggtctagagttgggatgagcattggtctctaatggttctgaaataattgtatattcctgcaaaaacattaagtctattagaaaccagctaatttcattttgtcatttttataggtaacatattctggtgcaggtagtatgtttttaaaacaagtttgcaataaacaatttcccctcaaggttaatataataggcaacaccttttgctgcaacagacggcaagaggtaatgaaagattagcttacattatgattcattatttcaaaatgtcaggataaagtggatctgctgcatctcccagagagtgcatgttttgcttttctaatgttaatggatttactgtttttttccccccaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggtaatagtgtgttgaaaacgacttctatttttgatcctatgagcagatcctaagagccaaagcgactgaggaaggaagacatagaatcagccatttgtacaaaacatgaatccctagtaggtccactagtatctttggtagaaacatggagaagagacaggatctcaggagaaggagttgacacatggcagggcagctgaggctgagtaattccgcttccttcctttggcaagactcaatcagtcttgagcaactctacagaagaattccactagctggatctctgaggaaaaaagaaatgttgtctgtgccctgactggggaatgccagatggacattcatgtttggtaggcaactttgcctatatgatctggtatatgctgttaattgtccatgcataattatctctctactcaggccttgtccaggcaaatattctgttttgttctagtttagcttgttctcccctttctctcttccatctctttcttgtctcaatggatgacaggatattttgctatgagctgactcagtggttggtgtcttgtaatggggagatatcatctttatcaaacagttattaagtatctacctgtagcatttcattttcccgcctgcctccattgttttcttgtctatagtttgccaattatagctaatatacggagagctatactttatttctactccagaaatgtctctattattgcattataataggataccctggggaaacactaatcatttttactacctaaaatacctatgctgaatatcctttatctgataggaacagagatctgacagcagcttaggctaaccaaattcattttttatcttaagtgtggggcatttttctctcttcttattctttaccttttcagcttaagtgaaggttagtataaacactaagaatatttctgatggagttttcatgtgattccttctacaaaaacccagatttaagtaacttgttgaaaaccagagtccgctaagttaataaacactgattgaagaagtgattctcatggactttctgtgatagctctttcctgccctgatatgagatgaaagctgggggatggtatatagtatttatttttccttccgttgccagtgggactttttttttttttttaaaagctgttcatatcttaatcgagtagcatgtgaggtcaacatggtctattttaaaagcattttcttcgacacattgcttttaacatcttttagaactctgctgtgagacacatggacttttttgttggtatttttatacaattaatgatattctcaatagtaatctttgtgtgtgtatatatatagaaataaattctaaatgtaagttaatatatttattatttttctaaacatatataaatatatatatgcacacaggctatttaatttta